SEQUENCE LISTING (3 sequences)

SEQ ID NO: 1 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 2 - protein, 44 aa, Homo sapiens
YADAIFTNSYRKVLGQLSARKLLQDIMSRQQGESNQERGARARL

SEQ ID NO: 3 - protein, 44 aa, Artificial Sequence; identical to SEQ ID NO: 2
  Synthetic polypeptide

(19) United States
(12) Patent Application Publication
(10) Pub. No.: US 2013/0302909 A1
(43) Pub. Date: Nov. 14, 2013

(54) ASSESSMENT OF IGF-1 LEVELS IN HIV-INFECTED SUBJECTS AND USES THEREOF

(71) Applicant: Diane Potvin, Montreal (CA)

(72) Inventor: Diane Potvin, Montreal (CA)

(73) Assignee: THERATECHNOLOGIES INC., Montreal, QC (CA)

(21) Appl. No.: 13/979,630

(22) PCT Filed: Jan. 11, 2012
(86) PCT No.: PCT/CA12/50012

§ 371 Date: Jul. 31, 2013

Related U.S. Application Data
(60) Provisional application No. 61/432,659, filed on Jan. 14, 2011.

(51) Int. Cl. G01N 33/74

(52) U.S. Cl. CPC G01N 33/74
USPC 436/501

(57) ABSTRACT

Improved methods for determining normal IGF-1 levels in HIV infected subjects, based on a determination of the log of IGF-1 values obtained in blood-derived samples from a population of HIV-infected subjects, are disclosed. Also disclosed are methods of determining whether a given HIV-infected subject exhibits a normal IGF-1 level, based on a comparison of either the IGF-1 value or the log of the IGF-1 value obtained from a blood-derived sample of the subject with a normative IGF-1 range determined using the exponentiation of the log of IGF-1 values or the log of IGF-values obtained in blood-derived samples from a population of age- and gender-matched HIV-infected subjects. Such methods are useful for example to monitor GH stimulation therapy in HIV-infected subjects.

### CROSS REFERENCE TO RELATED APPLICATIONS

This application claims the benefit of U.S. provisional application Ser. No. 61/432,659, filed on Jan. 14, 2011, which is incorporated herein by reference in its entirety.

### TECHNICAL FIELD

The present invention relates to the determination and monitoring of insulin-like growth factor 1 (IGF-1) levels in human immunodeficiency virus (HIV)-infected subjects.

### BACKGROUND ART

IGF-1 is a hormone of 70 amino acids produced primarily by the liver as an endocrine hormone as well as in target tissues in a paracrine/autocrine fashion. IGF-1 production is stimulated by growth hormone (GH); it is a primary mediator of the effects of GH. Analysis of serum/plasma IGF-1 level is commonly used alone or in combination with other criteria to evaluate pituitary function, in the diagnosis of GH-related disorders or in the monitoring/follow-up of patients receiving GH therapy, GH secretagogue therapy or GRF therapy.

Over the last decade, the importance of monitoring IGF-I levels in recipients of GH-related products has emerged as a key aspect of endocrine practice (Cohen P, et al., J Clin Endocrinol Metab. 2010; 95: 2089-98) and approaches to maintain levels at the upper limit of the normal range (+2 SDS) have been developed (Park P and Cohen P. Horm Res. 2004; 62: S59-65). Interpretation of IGF-1 levels is complicated by the wide normal ranges, and variations by age, sex, pubertal stage and diseases. Therefore, given the multiple facets of IGF-1 physiology and patho-physiology, the measurement of IGF-1 is not trivial.

Therefore, there is a need for the development of novel methods for determining and monitoring IGF-1 levels in subjects, and more particularly in subjects receiving GH therapy, GH secretagogue or GRF therapy, such as patients suffering from HIV-associated lipodystrophy.

The present description refers to a number of documents, the content of which is herein incorporated by reference in their entirety.

### SUMMARY OF THE INVENTION

The present invention relates to the determination and monitoring of insulin-like growth factor 1 (IGF-1) levels in human immunodeficiency virus (HIV)-infected subjects.

In a first aspect, the present invention provides a method for determining whether an HIV-infected subject has a normal insulin-like growth factor 1 (IGF-1) level, said method comprising determining an IGF-1 standard deviation score (SDS) based on an IGF-1 value obtained from a blood-derived sample from said HIV-infected subject, wherein said IGF-1 SDS is determined using the following equation:

IGF-1 SDS=(x−μ)/σ

in which
x is the log of said IGF-1 value;
μ is the mean of log of IGF-1 values obtained in blood-derived samples from a population of age- and gender-matched HIV-infected subjects; and
σ is the standard deviation of said log of IGF-1 values obtained in blood-derived samples from a population of age- and gender-matched HIV-infected subjects;
   wherein an IGF-1 SDS determined by said equation that is ≧−z_{α} or ≦z_{α} is indicative that said HIV-infected subject has a normal IGF-1 level, and wherein an IGF-1 SDS determined from the above equation that is <−z_{α} or >z_{α} is indicative that said HIV-infected subject has an abnormal IGF-1 level, wherein z_{α} is from 1.282 to 5.0.

The present invention further provides a method for determining whether an HIV-infected subject has a normal insulin-like growth factor 1 (IGF-1) level, said method comprising:
(a) obtaining an IGF-1 value from a blood-derived sample from said HIV-infected subject; and
(b) determining an IGF-1 standard deviation score (SDS) based on said IGF-1 value, wherein said IGF-1 SDS is determined using the following equation:

IGF-1 SDS=(x−μ)/σ
in which
   x is the log of said IGF-1 value;
   μ is the mean of log of IGF-1 values obtained in blood-derived samples from a population of age- and gender-matched HIV-infected subjects; and
   σ is the standard deviation of said log of IGF-1 values obtained in blood-derived samples from a population of age- and gender-matched HIV-infected subjects;
      wherein an IGF-1 SDS determined by said equation that is ≧−z_{α} or ≦z_{α} is indicative that said HIV-infected subject has a normal IGF-1 level, and wherein an IGF-1 SDS determined by said equation that is <−z_{α} or >z_{α} is indicative that said HIV-infected subject has an abnormal IGF-1 level, wherein z_{α} is from 1.282 to 5.0.

The present invention further provides a method for determining whether an HIV-infected subject has normal insulin-like growth factor 1 (IGF-1) level, said method comprising:
(a) determining or measuring the levels of IGF-1 in a blood-derived sample from said HIV-infected subject so as to obtain an IGF-1 value; and
(b) determining an IGF-1 standard deviation score (SDS) based on said IGF-1 value, wherein said IGF-1 SDS is determined using the following equation:

IGF-1 SDS=(x−μ)/σ
in which
   x is the log of said IGF-1 value;
   μ is the mean of log of IGF-1 values obtained in blood-derived samples from a population of age- and gender-matched HIV-infected subjects; and
   σ is the standard deviation of said log of IGF-1 values obtained in blood-derived samples from a population of age- and gender-matched HIV-infected subjects;
      wherein an IGF-1 SDS determined by said equation that is ≧−z_{α} or ≦z_{α} is indicative that said HIV-infected subject has a normal IGF-1 level, and wherein an IGF-1 SDS determined by said equation that is <−z_{α} or >z_{α} is indicative that said HIV-infected subject has an abnormal IGF-1 level, wherein z_{α} is from 1.282 to 5.0.

The present invention further provides a method for determining whether an HIV-infected subject has a normal insulin-like growth factor 1 (IGF-1) level, said method comprising:
(a) collecting a blood-derived sample from said HIV-infected subject;
(b) determining or measuring the levels of IGF-1 in said blood-derived sample so as to obtain an IGF-1 value; and
(c) determining an IGF-1 standard deviation score (SDS) based on said IGF-1 value, wherein said IGF-1 SDS is determined using the following equation:

IGF-1 SDS=(x−μ)/σ
in which
   x is the log of said IGF-1 value;
   μ is the mean of log of IGF-1 values obtained in blood-derived samples from a population of age- and gender-matched HIV-infected subjects; and
   σ is the standard deviation of said log of IGF-1 values obtained in blood-derived samples from a population of age- and gender-matched HIV-infected subjects;
      wherein an IGF-1 SDS determined by said equation that is ≧−z_{α} or ≦z_{α} is indicative that said HIV-infected subject has a normal IGF-1 level, and wherein an IGF-1 SDS determined by said equation that is <−z_{α} or >z_{α} is indicative that said HIV-infected subject has an abnormal IGF-1 level, wherein z_{α} is from 1.282 to 5.0.

The present invention further provides a method for determining whether an HIV-infected subject has a normal insulin-like growth factor 1 (IGF-1) level, said method comprising comparing a log transformed IGF-1 value obtained from a blood-derived sample from said HIV-infected subject to a log transformed normative range, wherein said log transformed normative range is determined using the following equation:

log transformed normative range=μ±(z_{α}·σ)

in which
μ is the mean of log transformed IGF-1 values obtained in blood-derived samples from a population of age- and gender-matched HIV-infected subjects;
z_{α} is from 1.282 to 5.0; and
σ is the standard deviation of said log of IGF-1 values obtained in blood-derived samples from a population of age- and gender-matched HIV-infected subjects;
   wherein a log transformed IGF-1 value that is within said log transformed normative range is indicative that said HIV-infected subject has a normal IGF-1 level, and a log transformed IGF-1 value that is outside said log transformed normative range is indicative that said HIV-infected subject has an abnormal IGF-1 level.

In an embodiment, the above-mentioned method further comprises determining or measuring the levels of IGF-1 in said blood-derived sample so as to obtain an IGF-1 value; and performing a log transformation of said IGF-1 value so as to obtain a log transformed IGF-1 value.

In another aspect, the present invention provides a method for determining whether an HIV-infected subject has a normal insulin-like growth factor 1 (IGF-1) level, said method comprising:
(a) obtaining an IGF-1 value from a blood-derived sample from said HIV-infected subject;
(b) performing a log transformation of said IGF-1 value to obtain a log transformed IGF-1 value; and
(c) comparing said log transformed IGF-1 value to a log transformed normative range, wherein said log transformed normative range is determined using the following equation:

log transformed normative range=μ±(z_{α}·σ)
in which
   μ is the mean of log transformed IGF-1 values obtained in blood-derived samples from a population of age- and gender-matched HIV-infected subjects;
   z_{α} is from 1.282 to 5.0; and
   σ is the standard deviation of said log of IGF-1 values obtained in blood-derived samples from a population of age- and gender-matched HIV-infected subjects;
      wherein a log transformed IGF-1 value that is within said log transformed normative range is indicative that said HIV-infected subject has a normal IGF-1 level, and a log transformed IGF-1 value that is outside said log transformed normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) determining or measuring the levels of IGF-1 in a blood-derived sample from said HIV-infected subject so as to obtain an IGF-1 value;
(b) performing a log transformation of said IGF-1 value to obtain a log transformed IGF-1 value; and
(c) comparing said log transformed IGF-1 value to a log transformed normative range, wherein said log transformed normative range is determined using the following equation:

log transformed normative range=μ±(z_{α}·σ)
in which
   μ is the mean of log transformed IGF-1 values obtained in blood-derived samples from a population of age- and gender-matched HIV-infected subjects;
   z_{α} is from 1.282 to 5.0; and
   σ is the standard deviation of said log of IGF-1 values obtained in blood-derived samples from a population of age- and gender-matched HIV-infected subjects;
      wherein a log transformed IGF-1 value that is within said log transformed normative range is indicative that said HIV-infected subject has a normal IGF-1 level, and a log transformed IGF-1 value that is outside said log transformed normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) collecting a blood-derived sample from said HIV-infected subject;
(b) determining or measuring the levels of IGF-1 in said blood-derived sample so as to obtain an IGF-1 value;
(c) performing a log transformation of said IGF-1 value to obtain a log transformed IGF-1 value; and
(d) comparing said log transformed IGF-1 value to a log transformed normative range, wherein said log transformed normative range is determined using the following equation:

log transformed normative range=μ±(z_{α}·σ)
in which
   μ is the mean of log transformed IGF-1 values obtained in blood-derived samples from a population of age- and gender-matched HIV-infected subjects;
   z_{α} is from 1.282 to 5.0; and
   σ is the standard deviation of said log of IGF-1 values obtained in blood-derived samples from a population of age- and gender-matched HIV-infected subjects;
      wherein a log transformed IGF-1 value that is within said log transformed normative range is indicative that said HIV-infected subject has a normal IGF-1 level, and a log transformed IGF-1 value that is outside said log transformed normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising comparing an IGF-1 value obtained from a blood-derived sample from said HIV-infected subject to a normative range, wherein said normative range is determined using the following equation:

normative range=A^{(μ±(Zα·σ))}

in which
μ is the mean of log_{A} transformed IGF-1 values obtained in blood-derived samples from a population of age- and gender-matched HIV-infected subjects;
A is the base of log_{A};
z_{α} is from 1.282 to 5.0; and
σ is the standard deviation of said log_{A} of IGF-1 values obtained in blood-derived samples from a population of age- and gender-matched HIV-infected subjects;
   wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) obtaining an IGF-1 value from a blood-derived sample from said HIV-infected subject; and
(b) comparing said IGF-1 value to a normative range, wherein said normative range is determined using the following equation:

normative range=A^{(μ±(Zα·σ))}
in which
   μ is the mean of log_{A} transformed IGF-1 values obtained in blood-derived samples from a population of age- and gender-matched HIV-infected subjects;
   A is the base of log_{A};
   z_{α} is from 1.282 to 5.0; and
   σ is the standard deviation of said log_{A} of IGF-1 values obtained in blood-derived samples from a population of age- and gender-matched HIV-infected subjects;
      wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) determining or measuring the levels of IGF-1 in said blood-derived sample so as to obtain an IGF-1 value; and
(b) comparing said IGF-1 value to a normative range, wherein said normative range is determined using the following equation:

normative range=A^{(μ±(Zα·σ))}
in which
   μ is the mean of log_{A} transformed IGF-1 values obtained in blood-derived samples from a population of age- and gender-matched HIV-infected subjects;
   A is the base of log_{A};
   z_{α} is from 1.282 to 5.0; and
   σ is the standard deviation of said log_{A} of IGF-1 values obtained in blood-derived samples from a population of age- and gender-matched HIV-infected subjects;
      wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) collecting a blood-derived sample from said HIV-infected subject;
(b) determining or measuring the levels of IGF-1 in said blood-derived sample so as to obtain an IGF-1 value; and
(b) comparing said IGF-1 value to a normative range, wherein said normative range is determined using the following equation:

normative range=A^{(μ±(Zα·σ))}
in which
   μ is the mean of log_{A} transformed IGF-1 values obtained in blood-derived samples from a population of age- and gender-matched HIV-infected subjects;
   A is the base of log_{A};
   z_{α} is from 1.282 to 5.0; and
   σ is the standard deviation of said log_{A} of IGF-1 values obtained in blood-derived samples from a population of age- and gender-matched HIV-infected subjects;
      wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

In another aspect, the present invention provides a method for determining a normative range for the monitoring of insulin-like growth factor 1 (IGF-1) levels in HIV-infected subjects, said method comprising:
(a) obtaining IGF-1 values from blood-derived samples from a population of HIV-infected subjects;
(b) calculating said normative range in accordance with the following equation:

normative range=A^{(μ±(Zα·σ))}
in which
   μ is the mean of log_{A} transformed IGF-1 values obtained in blood-derived samples from a population of HIV-infected subjects;
   A is the base of log_{A};
   z_{α} is from 1.282 to 5.0; and
   σ is the standard deviation of said log_{A} of IGF-1 values obtained in blood-derived samples from a population of HIV-infected subjects.

In another aspect, the present invention provides a method for determining a normative range for the monitoring of insulin-like growth factor 1 (IGF-1) levels in HIV-infected subjects, said method comprising:
(a) determining or measuring the levels of IGF-1 in blood-derived samples from HIV-infected subjects so as to obtain IGF-1 values;
(b) calculating said normative range in accordance with the following equation:

normative range=A^{(μ±(Zα·σ))}
in which
   μ is the mean of log_{A} transformed IGF-1 values obtained in blood-derived samples from a population of HIV-infected subjects;
   A is the base of log_{A};
   z_{α} is from 1.282 to 5.0; and
   σ is the standard deviation of said log_{A} of IGF-1 values obtained in blood-derived samples from a population of HIV-infected subjects.

In another aspect, the present invention provides a method for determining a normative range for the monitoring of insulin-like growth factor 1 (IGF-1) levels in HIV-infected subjects, said method comprising:
(a) collecting blood-derived samples from said HIV-infected subjects;
(b) determining or measuring the levels of IGF-1 in the blood-derived samples so as to obtain IGF-1 values;
(c) calculating said normative range in accordance with the following equation:

normative range=A^{(μ±(Zα·σ))}
in which
   μ is the mean of log_{A} transformed IGF-1 values obtained in blood-derived samples from a population of HIV-infected subjects;
   A is the base of log_{A};
   z_{α} is from 1.282 to 5.0; and
   σ is the standard deviation of said log_{A} of IGF-1 values obtained in blood-derived samples from a population of HIV-infected subjects.

In another aspect, the present invention provides a method of monitoring GH stimulation therapy in an HIV subject, comprising determining whether the subject exhibits a normal IGF-1 level using the methods described herein, wherein:
(a) a higher than normal IGF-1 level is indicative that the GH stimulation therapy is resulting in a GH level that is higher than a normal GH level;
(b) a lower than normal IGF-1 level is indicative that the GH stimulation therapy is resulting in a GH level that is lower than a normal GH level; and
(c) an IGF-1 level falling within the normal range is indicative that the GH stimulation therapy is resulting in a normal GH level.

In another aspect, the present invention provides a method of determining whether GH stimulation therapy of an HIV subject should be adjusted or modified, comprising determining whether the subject exhibits a normal IGF-1 level using the methods described herein, wherein:
(a) a higher than normal IGF-1 level is indicative that the GH stimulation therapy should be reduced, interrupted or stopped;
(b) a lower than normal IGF-1 level is indicative that the GH stimulation therapy should be increased; and
(c) an IGF-1 level falling within the normal range is indicative that the GH stimulation therapy may be maintained without any significant adjustment.

In an embodiment, the above-mentioned method further comprises adjusting or modifying the GH stimulation therapy in the subject undergoing GH stimulation therapy in accordance with the determination of whether the subject exhibits a normal IGF-1 level.

In an embodiment, the above-mentioned log or log_{A} is logₑ or log₁₀, in a further embodiment logₑ.

In embodiments, z_{α} is 1.645, 1.96, 2.0, 2.5, 2.576, or 3.0.

In an embodiment, A is e or 10, in a further embodiment e.

In an embodiment, the above-mentioned IGF-1 level is a serum IGF-1 level and said blood-derived sample is a serum sample.

In an embodiment, the above-mentioned HIV-infected subject suffers from lipodystrophy.

In an embodiment, the above-mentioned HIV-infected subject is undergoing a growth hormone stimulation therapy.

In another aspect, the present invention provides a program storage device readable by an electronic medium and tangibly storing instructions executable by the electronic medium to perform the method defined above.

In another aspect, the present invention provides a computer program product comprising a computer usable medium that tangibly stores as computer readable code instructions to perform the method defined above.

In another aspect, the present invention provides a use of (i) GH, (ii) a GH secretagogue, (iii) GRF, or a functional variant, analog and/or fragment of any of (i) to (iii), for the adjustment or modification of GH stimulation therapy in a subject undergoing GH stimulation therapy in accordance with a determination of whether the subject exhibits a normal IGF-1 level, wherein said determination is according to the method defined above.

In another aspect, the present invention provides a use of (i) GH, (ii) a GH secretagogue, (iii) GRF, or a functional variant, analog and/or fragment of any of (i) to (iii), for the preparation of a medicament for the adjustment or modification of GH stimulation therapy in a subject undergoing GH stimulation therapy in accordance with a determination of whether the subject exhibits a normal IGF-1 level, wherein said determination is according to the method defined above.

In another aspect, the present invention provides (i) GH, (ii) a GH secretagogue, (iii) GRF, or a functional variant, analog and/or fragment of any of (i) to (iii), for use in the adjustment or modification of GH stimulation therapy in a subject undergoing GH stimulation therapy in accordance with a determination of whether the subject exhibits a normal IGF-1 level, wherein said determination is according to the method defined above.

In another aspect, the present invention provides (i) GH, (ii) a GH secretagogue, (iii) GRF, or a functional variant, analog and/or fragment of any of (i) to (iii), for use in the preparation of a medicament for the adjustment or modification of GH stimulation therapy in a subject undergoing GH stimulation therapy in accordance with a determination of whether the subject exhibits a normal IGF-1 level, wherein said determination is according to the method defined above.

Other objects, advantages and features of the present invention will become more apparent upon reading of the following non-restrictive description of specific embodiments thereof, given by way of example only with reference to the accompanying drawings.

### DISCLOSURE OF INVENTION

In the studies described herein, it has been determined that HIV-infected subjects have a larger/broader normal range of serum IGF-1 levels and higher standard deviation (SD) scores relative to age and gender-matched healthy adults. It has further been determined in the studies described herein that the assessment of IGF-1 levels in HIV-infected subjects should be performed using a comparison with means and standard deviations derived from a population of age and gender-matched HIV-infected subjects. The studies described herein have also established that the determination of the normative range for IGF-1 levels in HIV-infected subjects involves the logarithmic transformation of the raw IGF-1 levels.

Accordingly, in a first aspect, the present invention provides a method for determining whether an HIV-infected subject has a normal insulin-like growth factor 1 (IGF-1) level, said method comprising determining an IGF-1 standard deviation score (SDS) based on an IGF-1 value obtained from a blood-derived sample from said HIV-infected subject, wherein said IGF-1 SDS is determined using the following equation:

IGF-1 SDS=(x−μ)/σ

in which

x is the log of said IGF-1 value;

μ is the mean of log of IGF-1 values obtained in blood-derived samples from a population of age- and gender-matched HIV-infected subjects; and

σ is the standard deviation of said log of IGF-1 values obtained in blood-derived samples from a population of age- and gender-matched HIV-infected subjects;

wherein an IGF-1 SDS determined by said equation that is ≧−z_{α} or ≦z_{α} is indicative said HIV-infected subject has a normal IGF-1 level, and wherein an IGF-1 SDS determined from the above equation that is <−z_{α} or >z_{α} is indicative that said HIV-infected subject has an abnormal IGF-1 level, wherein z_{α} is 1.282 or more, in an embodiment from 1.282 to 5.0. In further embodiments z_{α} is 1.282, 1.645, 1.96, 2.0, 2.5, 2.576, 3.0 or 5.0, in yet further embodiments, z_{α} is 1.645, 1.96, 2.0, 2.5, 2.576, or 3.0.

The present invention further provides a method for determining whether an HIV-infected subject has normal insulin-like growth factor 1 (IGF-1) level, said method comprising:
(a) obtaining an IGF-1 value from a blood-derived sample from said HIV-infected subject (e.g., by measuring IGF-1 levels in the sample using a conventional assay); and
(b) determining an IGF-1 standard deviation score (SDS) based on said IGF-1 value, wherein said IGF-1 SDS is determined using the following equation:

IGF-1 SDS=(x−μ)/σ
in which
   x is the log of said IGF-1 value;
   μ is the mean of log of IGF-1 values obtained in blood-derived samples from a population of age- and gender-matched HIV-infected subjects; and
   σ is the standard deviation of said log of IGF-1 values obtained in blood-derived samples from a population of age- and gender-matched HIV-infected subjects;
      wherein an IGF-1 SDS determined by said equation that is ≧−z_{α} or ≦z_{α} is indicative said HIV-infected subject has a normal IGF-1 level, and wherein an IGF-1 SDS determined by said equation that is <−z_{α} or >z_{α} is indicative that said HIV-infected subject has an abnormal IGF-1 level, wherein z_{α} is 1.282 or more, in an embodiment from 1.282 to 5.0. In a further embodiment z_{α} is 1.282, 1.645, 1.96, 2.0, 2.5, 2.576, 3.0 or 5.0, in yet a further embodiment, 1.645, 1.96, 2.0, 2.5, 2.576, or 3.0.

The present invention further provides a method for determining whether an HIV-infected subject has a normal insulin-like growth factor 1 (IGF-1) level, said method comprising comparing a log transformed IGF-1 value obtained from a blood-derived sample from said HIV-infected subject to a log transformed normative range, wherein said log transformed normative range is determined using the following equation:

log transformed normative range=μ±(z_{α}·σ)

in which
μ is the mean of log transformed IGF-1 values obtained in blood-derived samples from a population of age- and gender-matched HIV-infected subjects;
z_{α} is 1.282 or more, in an embodiment from 1.282 to 5.0, in a further embodiment z_{α} is 1.282, 1.645, 1.96, 2.0, 2.5, 2.576, 3.0 or 5.0, in yet a further embodiment, 1.645, 1.96, 2.0, 2.5, 2.576, or 3.0; and
σ is the standard deviation of said log of IGF-1 values obtained in blood-derived samples from a population of age- and gender-matched HIV-infected subjects;
   wherein a log transformed IGF-1 value that is within said log transformed normative range is indicative that said HIV-infected subject has a normal IGF-1 level, and a log transformed IGF-1 value that is outside said log transformed normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) obtaining an IGF-1 value from a blood-derived sample from said HIV-infected subject (e.g., by measuring IGF-1 levels in the sample using a conventional assay);
(b) performing a log transformation of said IGF-1 value to obtain a log transformed IGF-1 value; and
(c) comparing said log transformed IGF-1 value to a log transformed normative range, wherein said log transformed normative range is determined using the following equation:

log transformed normative range=μ±(z_{α}·σ)
in which
   μ is the mean of log transformed IGF-1 values obtained in blood-derived samples from a population of age- and gender-matched HIV-infected subjects;
   z_{α} is 1.282 or more, in an embodiment from 1.282 to 5.0, in a further embodiment z_{α} is 1.282, 1.645, 1.96, 2.0, 2.5, 2.576, 3.0 or 5.0, in yet a further embodiment, 1.645, 1.96, 2.0, 2.5, 2.576, or 3.0; and
   σ is the standard deviation of said log of IGF-1 values obtained in blood-derived samples from a population of age- and gender-matched HIV-infected subjects;
      wherein a log transformed IGF-1 value that is within said log transformed normative range is indicative that said HIV-infected subject has a normal IGF-1 level, and a log transformed IGF-1 value that is outside said log transformed normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising comparing an IGF-1 value obtained from a blood-derived sample from said HIV-infected subject to a normative range, wherein said normative range is determined using the following equation:

normative range=A^{(μ±(Zα·σ))}

(i.e. “exponentiation” of the μ±(z_{α}·σ) value when natural log transformation (logₑ) is used or 10 to the power of (μ±(z_{α}·σ)) when log₁₀ is used)

in which
μ is the mean of log_{A} transformed IGF-1 values obtained in blood-derived samples from a population of age- and gender-matched HIV-infected subjects;
A is the base of log_{A};
z_{α} is 1.282 or more, in an embodiment from 1.282 to 5.0, in a further embodiment z_{α} is 1.282, 1.645, 1.96, 2.0, 2.5, 2.576, 3.0 or 5.0, in yet a further embodiment, 1.645, 1.96, 2.0, 2.5, 2.576, or 3.0; and
σ is the standard deviation of said log_{A} of IGF-1 values obtained in blood-derived samples from a population of age- and gender-matched HIV-infected subjects;
   wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

In another aspect, the present invention provides a method for determining whether an HIV-infected subject has a normal insulin-like growth factor 1 (IGF-1) level, said method comprising:
(a) obtaining an IGF-1 value from a blood-derived sample from said HIV-infected subject (e.g., by measuring IGF-1 levels in the sample using a conventional assay); and
(b) comparing said IGF-1 value to a normative range, wherein said normative range is determined using the following equation:

normative range=A^{(μ±(Zα·σ))}

(i.e. “exponentiation” of the μ±(z_{α}·σ) value when natural log transformation (logₑ) is used or 10 to the power of (μ±(z_{α}·σ)) when log₁₀ is used)
in which
   μ is the mean of log_{A} transformed IGF-1 values obtained in blood-derived samples from a population of age- and gender-matched HIV-infected subjects;
   A is the base of log_{A};
   z_{α} is 1.282 or more, in an embodiment from 1.282 to 5.0, in a further embodiment z_{α} is 1.282, 1.645, 1.96, 2.0, 2.5, 2.576, 3.0 or 5.0, in yet a further embodiment, 1.645, 1.96, 2.0, 2.5, 2.576, or 3.0; and
   σ is the standard deviation of said log_{A} of IGF-1 values obtained in blood-derived samples from a population of age- and gender-matched HIV-infected subjects;
      wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

In another aspect, the present invention provides a method for determining a normative range for the monitoring of insulin-like growth factor 1 (IGF-1) levels in HIV-infected subjects, said method comprising:
(a) obtaining IGF-1 values from blood-derived samples from a population of HIV-infected subjects (e.g., by measuring IGF-1 levels in the sample using a conventional assay);
(b) calculating said normative range in accordance with the following equation:

normative range=A^{(μ±(Zα·σ))}

(i.e. “exponentiation” of the μ±(z_{α}·σ) value when natural log transformation (logₑ) is used or 10 to the power of (μ±(z_{α}·σ)) when log₁₀ is used)
in which
   μ is the mean of log_{A} transformed IGF-1 values obtained in blood-derived samples from a population of HIV-infected subjects;
   A is the base of log_{A};
   z_{α} is 1.282 or more, in an embodiment from 1.282 to 5.0, in a further embodiment z_{α} is 1.282, 1.645, 1.96, 2.0, 2.5, 2.576, 3.0 or 5.0, in yet a further embodiment, 1.645, 1.96, 2.0, 2.5, 2.576, or 3.0; and
   σ is the standard deviation of said log_{A} of IGF-1 values obtained in blood-derived samples from a population of HIV-infected subjects.

Logarithmic transformation or log transformation of raw IGF-1 values (i.e. measured in blood-derived samples from subjects) can be done using different log bases. In embodiments, the above-mentioned log base is log base 10 (log₁₀) or natural log(logₑ or ln), in a further embodiment logₑ. The number e (sometimes called Euler's number) is an irrational constant well known in the mathematics field and is approximately equal to 2.718281828459.

The normative range is determined using the formula: A^{μ±(Zα·σ))}. Therefore, it should be understood that the lower limit of the normative range corresponds to: A^{μ-(Zα·σ))}, and the upper limit of the normative range corresponds to A^{μ+(Zα·σ))}. Similarly, the log transformed normative range is determined using the formula: μ±(z_{α}·σ). Therefore, it should be understood that the lower limit of the log transformed normative range corresponds to: μ−(z_{α}·σ), and the upper limit of the log transformed normative range corresponds to: μ+(z_{α}·σ).

“Abnormal” IGF-1 level as referred to herein may be lower or higher than normal IGF-1 level. For example, an IGF-1 SDS that is <−z_{α} is indicative of a lower than normal IGF-1 level, and an IGF-1 SDS that is >z_{α} is indicative of a higher than normal IGF-1 level.

z_{α} estimates the (1−α) percentile of a normally distributed population (i.e. probability that x<z_{α}=1−α, where x is log of IGF-1 value). z_{α} determines a normative range for comparing the IGF-1 values of specific HIV-infected subjects. For example, z_{α}=1.282 estimates the 90 percentile of the population, z_{α}=1.645 estimates the 95 percentile, z_{α}=1.96 estimates the 97.5 percentile, z_{α}=2.0 estimates the 97.7 percentile, z_{α}=2.5 estimates the 99.4 percentile, z_{α}=2.576 estimates the 99.5 percentile, z_{α}=3.0 estimates the 99.9 percentile and z_{α}=5 estimates the 99.99999 percentile. In embodiments, z_{α} is 1.282 or more; 1.645 or more; 1.96 or more; 2.0 or more; 2.5 or more; 2.576 or more; or 3.0 or more. In embodiments z_{α} is from 1.282 to 5.0; 1.645 to 5.0; 1.96 to 5.0; 2.0 to 5.0; 2.5 to 5.0; 2.576 to 5.0; 1.282 to 3.0; 1.645 to 3.0; 1.96 to 3.0; 2.0 to 3.0; 1.282 to 2.5; 1.645 to 2.5; 1.96 to 2.5; or 2.0 to 2.5. In further embodiments, z_{α} is 1.282, 1.645, 1.96, 2.0, 2.5, 2.576, 3.0 or 5.0, in yet further embodiments 1.645, 1.96, 2.0, 2.5, 2.576, or 3.0. In an embodiment, z_{α} is 1.282. In another embodiment, z_{α} is 1.645. In another embodiment, z_{α} is 1.96. In another embodiment, z_{α} is 2.0. In another embodiment, z_{α} is 2.5. In another embodiment, z_{α} is 2.576. In another embodiment, z_{α} is 3.0. In another embodiment, z_{α} is 5.0.

The methods of the invention may be repeated (e.g., 2, 3, 4, 5 or more times) in order to compare more than once with the normative range or the log transformed normative range or to calculate the IGF-1 SDS of a HIV-infected subject in order to verify the determination/conclusion of abnormal IGF-1 level versus normal IGF-1 level.

In an embodiment, the above-mentioned methods further comprise measuring (or determining, analyzing, quantifying) IGF-1 levels in a sample from a subject to obtain an IGF-1 value.

IGF-1 levels/values may be measured or determined using well known standard methods for measuring polypeptide levels. The amino acid sequence of mature human IGF-1 polypeptide is depicted in SEQ ID NO:1. Non-limiting examples of such methods include Western blot, immunoblot, enzyme-linked immunosorbant assay (ELISA), radioimmunoassay (RIA, e.g., blocking or competitive RIA), immunoprecipitation, surface plasmon resonance (SPR), chemiluminescence, fluorescent polarization, phosphorescence, matrix-assisted laser desorption/ionization time-of-flight (MALDI-TOF) mass spectrometry, Liquid chromatography-mass spectrometry (LCMS)-MS mass spectrometry, microcytometry, microscopy, and flow cytometry. Kits for determining IGF-1 levels in biological samples are commercially available, for example from Enzo™ Life Sciences (Cat. No. ADI-900-150), AbFrontier™ Co., Ltd (Cat. No. LF-EK50092), Abnova™ (Cat. No. KA0349), R&D Systems™ (Cat. No. DG100) and others. In an embodiment, the above-mentioned methods comprise contacting the sample with an agent that binds to IGF-1 (e.g., an anti-IGF-1 antibody or an antigen-binding fragment thereof, an IGF-1 ligand/binding partner or an analog thereof), and measuring/determining the level of binding of said agent to the IGF-1 present in the sample. The levels of IGF-1 in the sample can be determined/evaluated by comparison with values obtained in one or more control or reference samples (in which a known, pre-determined amount of IGF-1 is present). In an embodiment, the IGF-1 levels/values are measured/determined by radioimmunoassay (RIA), for example using the blocking/competitive RIA assay described in Example 1 below.

In an embodiment, the above-mentioned methods further comprise collecting a blood-derived sample from a subject, optionally treating the blood-derived sample, and measuring/determining IGF-1 levels in the sample to obtain an IGF-1 value.

“Blood-derived sample” as used herein refers to whole blood or to a fraction thereof, such as serum or plasma. It also refers to any sample that may be obtained following one or more purification/enrichment steps or any other treatment using whole blood (obtained by venous puncture, for example) as starting material. In an embodiment, the above-mentioned blood-derived sample is serum. In an embodiment, the above-mentioned blood-derived sample is a fresh sample (which has not been subjected to storage, freezing, etc.). In another embodiment, the above-mentioned blood-derived sample is frozen prior to the determination of IGF-1 levels. In an embodiment, the blood-derived sample (e.g., serum sample) is treated prior to the determination of IGF-1 levels. In a further embodiment, the treatment comprises an acid/ethanol extraction to separate serum IGF-1 from binding proteins.

“Standard deviation score” (SDS) (also called standard score, z-value, z-score, normal score, and standardized variable) indicates how many standard deviations an observation or datum is above or below the population mean. It is obtained by subtracting the population mean μ (in the present case, the log (e.g., logₑ or log₁₀) of the IGF-1 value obtained in blood-derived sample from a population of age- and gender-matched HIV-infected subjects) from an individual raw score x (in the present case, the log (e.g., logₑ or log₁₀) of the IGF-1 value obtained in a blood-derived sample from the HIV-infected subject under testing) and then dividing the difference by the population standard deviation a. Therefore, a SDS of −2.0 indicates that the raw data is equivalent to: [(mean of the population)−(2 times the standard deviation)], whereas a SDS of +2.0 indicates that the raw data is equivalent to: [(mean of the population)+(2 times the standard deviation)].

In an embodiment, the above-mentioned HIV-infected subject suffers from lipodystrophy (typically referred to as HIV lipodystrophy, HIV-associated lipodystrophy or HIV-related lipodystrophy). “Lipodystrophy” refers to a condition characterized by abnormal or degenerative conditions of the body's adipose tissue, and more particularly to abnormal lipid production, distribution, storage and/or metabolism, with excess, or lack of, fat in various regions of the body.

In an embodiment, the above-mentioned HIV-infected subject is undergoing growth hormone (GH) stimulation therapy. The term “GH stimulation therapy” refers to any treatment aimed at increasing the levels of GH in a subject, either directly or indirectly, and in embodiments refers to “GH therapy”, “GH secretagogue therapy” or “GRF therapy”.

“GH therapy” refers to the administration of GH (e.g., through the administration of recombinant GH), or an analog thereof or variant thereof that retains the biological activity of native GH. GH analogs or variants are well known in the art (see, e.g., WO/2009/156511, US 20040142870, WO/2000/015664, WO/2010/084173, WO/2007/07742, WO/1997/011178, and WO/2006/042848)

“GH secretagogue therapy” refers to the administration any GH secretagogue which binds a growth hormone secretagogue receptor (e.g., a ghrelin receptor) and stimulates GH release (e.g. ghrelin, or an analog thereof that retains the biological activity of native ghrelin). Agonists of the ghrelin receptors are well known in the art (see, e.g., WO/2008/148854, WO/2001/092292, WO/2004/009616, WO/2008/092681, WO/2009/140763, WO/2006/009674, and WO/2007/020013).

“GRF therapy” refers to the administration of growth-hormone-releasing factor (GRF) (also known as growth-hormone-releasing hormone, GHRH), an analog thereof, a variant thereof or a variant of an analog thereof which binds a GRF receptor and stimulate GH release, such as GRF or an analog thereof (such as (hexenoyl trans-3)hGRF₍₁₋₄₄₎NH₂).

The mature active human GRF is a 44 amino acid peptide having the following structure (see, e.g., UniProtKB Accession No. P01286):

(SEQ ID NO: 2)
Tyr-Ala-Asp-Ala-Ile-Phe-Thr-Asn-Ser-Tyr-Arg-Lys-Val-Leu-Gly-Gln-Leu-Ser-Ala-Arg-
Lys-Leu-Leu-Gln-Asp-Ile-Met-Ser-Arg-Gln-Gln-Gly-Glu-Ser-Asn-Gln-Glu-Arg-Gly-Ala-
Arg-Ala-Arg-Leu.

The terms GHRH, GRF or an analog thereof, a variant thereof or a variant of an analog thereof which binds a GRF receptor and stimulate GH release, as used herein, include, without limitation, human native GRF (1-44) and fragments thereof (1-40), (1-29), fragments ranging between 1-29 and the 1-44 of the GRF peptide sequence, and any other fragments that possess agonist activity for a GHRH receptor and/or activity of stimulating GH secretion; GRF from other species and fragments thereof that possess agonist activity for a GHRH receptor and/or activity of stimulating GH secretion; GRF variants containing amino acid(s) substitution(s), addition(s) and/or deletion(s) for example variants having at least about 50, 55, 60, 65, 70, 75, 80, 85, 90%, 95% or more of similarity or identity with the native amino acid sequence and/or with the GRF(1-29) fragment, and which possess agonist activity for a GHRH receptor and/or activity of stimulating GH secretion. In an embodiment, the above-mentioned fragments/variants retain at least about 10%, 20%, 30%, 40%, 50%, 60%, 70%, 75%, 80%, 85%, 90% or 95% of the activity (e.g., agonist activity for a GHRH receptor and/or activity of stimulating GH secretion) of the native GRF. These terms as used herein also encompass derivatives or analogs of GRF or fragments or variants thereof having for a example an organic group or a moiety coupled to the GRF amino acid sequence at the N-terminus, the C-terminus and/or on a side-chain; and salts of GRF (human or from other species), as well as salts of GRF fragments, variants, analogs and derivatives. Also encompassed are GRF molecules currently known in the art, including, without limitation, albumin-conjugated GHRH (U.S. Pat. No. 7,268,113); pegylated GHRH peptides (U.S. Pat. Nos. 7,256,258 and 6,528,485); porcine GHRH (1-40) (U.S. Pat. No. 6,551,996); canine GHRH (U.S. patent application no. 2005/0064554); GHRH variants of 1-29 to 1-44 amino acid length (U.S. Pat. Nos. 5,846,936, 5,696,089, 5,756,458 and 5,416,073, and U.S. patent application Nos. 2006/0128615 and 2004/0192593); and Pro⁰-GHRH peptide and variants thereof (U.S. Pat. No. 5,137,872).

The GRF analogs also include those described in U.S. Pat. Nos. 5,861,379, 5,939,386, 6,020,311 and 6,458,764 which also describe their method of synthesis.

Compositions comprising a GRF analog are described in for example published U.S. patent application No. 20080249017.

One of the mechanisms by which GH stimulation therapies mediate their biological/therapeutic effects is through the induction of IGF-1 production, and thus IGF-1 levels constitute a good marker for monitoring GH stimulation therapy. Therefore, the above-mentioned methods, which allow for more reliable/precise determination of the normative range of IGF-1 levels (i.e., what constitutes “normal” and “abnormal” levels) in the HIV-infected population, may be useful to monitor the efficacy of GH stimulation therapy (e.g., GH therapy, GRF therapy or GH secretagogue therapy), and more particularly to determine whether the levels of IGF-1 in the subject undergoing GH stimulation therapy are normal or abnormal. If a subject undergoing GH stimulation therapy exhibits higher than normal IGF-1 levels (more than +z_{α} SDS), the dose of the agent administered may be adjusted or the treatment could be reduced or interrupted, for example.

As such, the present invention further provides a method of monitoring GH stimulation therapy in an HIV subject, comprising determining whether the subject exhibits a normal IGF-1 level using the method described herein, wherein:
(a) a higher than normal IGF-1 level is indicative that the GH stimulation therapy is resulting in a GH level that is higher than a normal GH level;
(b) a lower than normal IGF-1 level is indicative that the GH stimulation therapy is resulting in a GH level that is lower than a normal GH level; and
(c) an IGF-1 level falling within the normal range is indicative that the GH stimulation therapy is resulting in a normal GH level.

The present invention further provides a method of determining whether GH stimulation therapy of an HIV subject should be adjusted or modified, comprising determining whether the subject exhibits a normal IGF-1 level using the method described herein, wherein:
(a) a higher than normal IGF-1 level is indicative that the GH stimulation therapy should be reduced, interrupted or stopped;
(b) a lower than normal IGF-1 level is indicative that the GH stimulation therapy should be increased; and
(c) an IGF-1 level falling within the normal range is indicative that the GH stimulation therapy may be maintained without any significant adjustment.

In an embodiment, the above noted method further comprises adjusting or modifying the GH stimulation therapy in the subject in accordance with the above noted determination of IGF-1 level in the subject undergoing GH stimulation therapy. For example, in the case of a higher than normal IGF-1 level, the GH stimulation therapy may be reduced, for example by reducing the dosage and/or frequency of administration of the GH stimulation therapy (e.g., GH therapy, GRF therapy or GH secretagogue therapy), interrupting the therapy for a period of time or halting it altogether, or by otherwise altering the therapy (e.g., by using a different GH stimulating agent) so that it results in lower GH levels. In an embodiment, the above-mentioned method comprises administering a lower dose of medicament (e.g., GH, GRF, GH secretatogue, or an analog thereof), and/or administering said medicament at a lower frequency, relative to the dose and/or frequency of administration prior to the above-mentioned determination.

Similarly, in the case of a lower than normal IGF-1 level, the GH stimulation therapy may be increased, for example by increasing the dosage and/or frequency of administration of the GH stimulation therapy (e.g., of GH, GRF or GH secretagogue, or analogs thereof) or by otherwise altering the therapy (e.g., by using a different GH stimulating agent, possibly in combination with the GH stimulating agent already being used) so that it results in higher GH levels. In an embodiment, the above-mentioned method comprises administering a higher dose of medicament (e.g., GH, GRF, GH secretatogue, or an analog thereof), and/or administering said medicament at a lower frequency, relative to the dose and/or frequency of administration prior to the above-mentioned determination.

In an embodiment, one or more steps of the above-mentioned methods are performed using or by a computer (e.g., using computer algorithms). For example, the calculation of the log value based on sampled IGF-1 levels, the determination of the mean and standard deviation based on data collected from a population of HIV-infected subjects, the determination of the normative range and/or log transformed normative range, the determination of the IGF-1 standard deviation score (SDS), and/or the comparison of the IGF-1 value to the normative range, may be performed using a suitably programmed computer.

According to various embodiments, the method can further comprise sampling the blood of an HIV-infected subject and determining the IGF-1 level of the sampled blood or a fraction thereof. In an embodiment, the sampled IGF-1 level can subsequently be stored in a computer in a suitable computer readable form. The computer can subsequently be used to to transform the sampled IGF-1 level using a logarithmic transformation and calculate an IGF-1 SDS using the mean, standard deviation, and also to compare the IGF-1 level to a normative range, etc. of the IGF-1 levels of the population of HIV-infected subjects. The data or results can then be displayed, for example, on a monitor, and/or printed.

In embodiments, the methods further comprise transmitting the data or results over a communication network. For example, the data or results may be transferred from a laboratory testing facility (e.g., diagnostic laboratory) to a health care provider, who may analyse the data/results and/or choose the appropriate course of action based on the data/results (e.g., continue therapy, interrupt therapy, modify the dosage regimen, etc.).

In another aspect, the present invention provides a program storage device readable by an electronic medium and tangibly storing instructions executable by the electronic medium to perform the above-mentioned methods.

In another aspect, the present invention provides a computer program product comprising a computer useable medium that tangibly stores as computer readable code instructions to perform the above-mentioned methods.

### MODE(S) FOR CARRYING OUT THE INVENTION

The present invention is illustrated in further details by the following non-limiting examples.

### Example 1

### Materials and Methods

The IGF-1 normative ranges were derived from two phase 3 clinical studies of treatment with the GRF analog (hexenoyl trans-3)hGRF(1-44)NH₂ (also referred to herein as “ThGRF” or “TH9507”). One phase 3 clinical study includes a main phase (6 months) and an extension phase (6 months) in one study (Lipo-010) and the second phase 3 clinical study includes two studies. CTR-1011 was the main phase (first 6 months) and CTR-1012 was the extension phase (last 6 months). The description of the phase 3 clinical studies is provided in Table I below:

TABLE I
Description of the phase 3 clinical studies

| | | | Subjects |
|---|---|---|---|
| | | | Safety Population (Gender) |
| Study and | | | Mean age (range) |
| Study Design | Study Objectives | Treatment | Race |
| TH9507/III/LIPO/010 | Main phase (Weeks 0-26) (1) | Main phase | Main phase |
| Multicenter (43 | Demonstrate a reduction in | TH9507, 2 mg/day; | 410 (352 M, 58 F) |
| centers; Canada, US), | VAT after 26 weeks of | matching Placebo | 48 (28-65) y |
| randomized, double- | treatment with TH9507 as | Extension phase | White 75.1%, |
| blind, placebo- | compared to placebo. (2) | TH9507, 2 mg/day | Black 14.4%, |
| controlled, parallel- | Demonstrate the efficacy of | In subjects who | Hispanic 8.3%, |
| group | TH9507 as compared to | received TH9507 in | Asian 0.5%, |
| | placebo after 26 weeks of | the main phase: | Other 1.7% |
| | treatment as evidenced by an | TH9507-TH9507 | Extension phase |
| | improvement in: TC:HDL-C | sequence (T-T group) | 315 (275 M, 40 F) |
| | ratio and TG levels, IGF-1 | In subjects who | 48 (29-65) y |
| | levels, and PRO related to | received placebo in | White 77.5%, |
| | body image. (3) Demonstrate | the main phase: | Black 12.4%, |
| | the safety of TH9507 as | Placebo-TH9507 | Hispanic 7.9%, |
| | compared to placebo after 26 | sequence (P-T group) | Asian 0.6%, |
| | weeks of treatment. | Matching Placebo | Other 1.6% |
| | Extension phase (Weeks 27- | In subjects who | |
| | 52) (1) Assess the long-term | received TH9507 in | |
| | safety of TH9507 (26 weeks in | the main phase: | |
| | patients who received placebo | TH9507-Placebo | |
| | in the main phase [P-T group]; | sequence (T-P group) | |
| | 52 weeks in patients who | | |
| | received TH9507 in the main | | |
| | phase [T-T group]). (2) | | |
| | Assess, over a 26-week | | |
| | placebo treatment period, the | | |
| | duration of effect on VAT and | | |
| | lipid profile following 26 weeks | | |
| | of treatment with TH9507 as | | |
| | compared to baseline and | | |
| | Week 26 (T-P group). (3) | | |
| | Collect data on efficacy after | | |
| | 52 weeks of treatment with | | |
| | TH9507. | | |
| TH9507-CTR-1011 | (1) Evaluate the reduction in | TH9507, 2 mg/day; | 396 (333 M, 63 F) |
| Multicenter (48 | VAT after 26 weeks of | matching Placebo | 48 (27-65) y |
| centers; Canada, US, | treatment with TH9507 | | White 77.0%, |
| UK, Belgium, Spain, | compared to placebo. (2) | | Black 11.6%, |
| France), randomized, | Evaluate the efficacy of | | Hispanic/Latino 8.8%, |
| double-blind, placebo- | TH9507 as compared to | | Asian 0.8%, |
| controlled, parallel- | placebo after 26 weeks of | | Other 1.8% |
| group | treatment by measuring an | | |
| | improvement in PRO related | | |
| | to body image, TC:HDL-C | | |
| | ratio and TG levels; IGF-1 | | |
| | concentrations. (3) Evaluate | | |
| | the safety of TH9507 as | | |
| | compared to placebo after | | |
| | 26 weeks of treatment. | | |
| TH9507-CTR-1012 | (1) Assess the long-term | TH9507, 2 mg/day | 263 (234 M, 29 F) |
| Multicenter (40 | safety of 2 mg daily doses of | In subjects who | 48 (29-65) y |
| centers; Canada, US, | subcutaneous (sc) TH9507 | received TH9507 in | White 82.9%, |
| UK, Belgium, Spain, | (26 weeks in a population | the TH9507-CTR-1011 | Black/African American 8.0%, |
| France), randomized, | previously receiving placebo in | study: TH9507-TH9507 | Hispanic 7.2%, |
| double-blind, placebo- | the TH9507-CTR-1011 study | sequence (T-T group) | Other 2.0% |
| controlled, parallel- | [P-T group]; 52 weeks for a | In subjects who | |
| group | subgroup of patients | received placebo in | |
| | previously receiving TH9507 in | the TH9507-CTR-1011 | |
| | theTH9507-CTR-1011 study | study: Placebo-TH9507 | |
| | [T-T group]. (2) Assess over a | sequence (P-T group) | |
| | 26-week placebo treatment | Matching Placebo | |
| | period, the duration of effect of | In subjects who | |
| | TH9507 on VAT, trunk fat and | received TH9507 in | |
| | lipid profile following a | the TH9507-CTR-1011 | |
| | 26-week treatment with 2 mg | study: TH9507-Placebo | |
| | daily doses of sc TH9507 (T-P | sequence (T-P group) | |
| | group). (3) Collect data on | | |
| | efficacy after a 52-week | | |
| | treatment with TH9507. | | |

The IGF-1 data at baseline of all male patients from studies Lipo-010 and CTR-1011 were combined. Logₑ transformation was applied. Age-specific means and standard-deviations were calculated from these data and normative ranges calculated. The level of IGF-1 in the serum samples was determined using a blocking RIA assay. The IGF-1 blocking RIA assay is a competitive binding radioimmunoassay. It utilizes rabbit polyclonal antisera specific for IGF-1 as the primary antibody and radioiodinated recombinant human IGF-1 as tracer. Serum IGF-1 is first separated from binding proteins by acid ethanol extraction. The extracts (controls and unknown/test samples) and standard are incubated with primary antibody and tracer. Free IGF-1 present in the extracts competes with and displaces the tracer from primary antibody. A second antibody specific for rabbit immunoglobulin is added to precipitate the immunocomplexes, which are then separated from free reactants by centrifugation. The amount of radioactivity present in the precipitated immunocomplex is measured with a gamma counter and is inversely related to the concentration of IGF-1 present in the sample. The amount of tracer bound to the immunocomplexes formed in the calibrator samples (purified recombinant human IGF-1) is used to establish a dose response standard curve from which the IGF-1 concentration of controls and unknown samples are back calculated.

### Example 2

### Defining the Normative Ranges and Standard Deviation Score (SDS) Calculations for Serum IGF-I in Adults with HIV

The assessment of IGF-1 levels was utilized to monitor clinical studies of the treatment of HIV subjects with the GRF analog (hexenoyl trans-3)hGRF(1-44)NH₂. (hexenoyl trans-3)hGRF(1-44)NH₂, also referred to herein as “ThGRF” or “TH9507”, has the following structure:

(SEQ ID NO: 3)
(trans)CH₃—CH₂—CH═CH—CH₂—CO-Tyr-Ala-Asp-Ala-Ile-Phe-Thr-Asn-Ser-Tyr-Arg-Lys-Val-
Leu-Gly-Gln-Leu-Ser-Ala-Arg-Lys-Leu-Leu-Gln-Asp-Ile-Met-Ser-Arg-Gln-Gln-Gly-Glu-Ser-
Asn-Gln-Glu-Arg-Gly-Ala-Arg-Ala-Arg-Leu-NH₂

During clinical studies of the treatment of HIV subjects with ThGRF, initial analyses of IGF-I values of the HIV subjects was performed using a typical IGF-I determination, in which age- and gender-specific means and SD values were utilized for the calculation of the standard deviation scores (SDS). These values were derived from a relatively small cohort of healthy adults and were established assuming a Normal distribution (without any transformation of the measured IGF-1 values). As such, determination of the normative IGF-1 range was initially performed based on age- and gender-matched healthy (i.e., non-HIV) subjects, without any transformation of the measured raw IGF-1 values, referred to herein below as the “typical method” or “typical approach”.

Based on the assumed normal distribution, it was expected that +2 SDS will estimate the 97.7 percentile and that only 2.3% of subjects will have SDS values higher than +2 SDS, and about 0.1% of subjects will have values larger than +3 SDS, in the untreated state (i.e., patients at baseline prior to ThGRF treatment, or placebo treated patients). It was also expected to see the standard deviations of IGF-1 SDS to be equal to 1.0.

However as seen in Table II below, by using the typical approach for IGF-1 determination described above, the observed values for the percent of patients at baseline as well as placebo patients throughout the studies exceeded the expected percentages of 2.3% by more than two-fold, with about 6% of patients having values larger than +2 SDS at baseline and 5% of placebo patients being larger than +2 SDS at Week 26. In addition, the expected percentage of patients exceeding SDS >+3, which should be 0.1%, was significantly higher than expected (with 3.8% and 2.5% of patients being above +3 SDS at baseline and placebo Week 26, respectively). Furthermore, the standard deviation of IGF-1 SDS values, when calculated in the trials exceeded the expected value of 1.0 (with standard deviation of 1.54 at baseline and of 1.26 for placebo patients at Week 26).

TABLE II
IGF-1 Level and SDS at Baseline and Week 26, Combined
Phase 3 Studies, determined using typical approach

| | | LIPO-010 Main/CTR-1011 | |
|---|---|---|---|
| | | ThGRF | |
| | | (2 mg/day) | Placebo |
| | | (N = 543) | (N = 263) |
| BASELINE | | | |
| IGF-1 level (ng/ml) | n | 534 | 261 |
| | Mean (SD) | 154 (62.9) | 159 (68.5) |
| IGF-1 SDS | n | 534 | 261 |
| | Mean (SD) | −0.3 (1.32) | −0.2 (1.54) |
| SDS >+2 (n, %) | | 33 (6.2) | 16 (6.1) |
| SDS >+3 (n, %) | | 8 (1.5) | 10 (3.8) |
| WEEK 26 | | | |
| IGF-1 level (ng/ml) | n | 534 | 261 |
| | Mean (SD) | 262 (123.6) | 152 (65.1) |
| IGF-1 SDS (SD) | n | 405 | 202 |
| | Mean (SD) | 2.4 (2.85) | −0.5 (1.26) |
| SDS >+2 (n, %) | | 192 (47.4) | 10 (5.0) |
| SDS >+3 (n, %) | | 144 (35.6) | 5 (2.5) |

SD = standard deviation, SDS = standard deviation score

As can be seen in Table III below, the mean IGF-1 levels of healthy subjects are comparable to the means of the HIV patients of the clinical studies. However, in older groups (41-50, 51-60 and 61-70), the standard deviations seem to be substantially larger even though the means were similar. In fact, using the initial method of IGF-1 determination noted above, the standard deviations are about 50% larger for the same age groups.

TABLE III
IGF-1 Reference Intervals for HIV⁺ and HIV⁻ Male Subjects

| | | |
|---|---|---|
| | HIV−^{a} | HIV+^{b} |
| Age (years) | Mean (SD) | Mean (SD) |

| | | | |
|---|---|---|---|
| 21-30 | 289 (73) | | |
| | | {close oversize brace} | 176 (65)^{c} |
| 31-40 | 226 (62) | | |
| 41-50 | 160 (42) | | 154 (62) |
| 51-60 | 153 (48) | | |
| | | {close oversize brace} | 148 (63)^{d} |
| 61-70 | 132 (34) | | |

SD = standard deviation
^{a}Based on non-HIV-infected, healthy subjects
^{b}Based on values from HIV⁺ patients in the Phase 3 studies (LIPO-010, CTR-1011, and CTR-1012)
^{c}Age category is 21-40 years
^{d}Age category is 51-70 years

As such, the normative ranges provided by the typical method of IGF-1 determination noted above appears to significantly overestimate the percentage of HIV patients having scores higher than +2 SDS and +3 SDS. In fact, it seems that the +3 SDS derived using the normative ranges provided by the typical method noted above represents more or less the upper limit of normal (97.5%) of the HIV population.

The IGF-1 SDS were recalculated based on the means and standard deviations calculated from HIV patients (from all male patients in the above-noted clinical studies at baseline per age group as presented in Table III above, assuming a Normal distribution). The results are presented in Table IV for the main phase and the extension phase. The percentages of patients larger than +2 and +3 SDS are lower using this approach, with 41.2% and 25.4% of ThGRF-treated patients larger than +2 SDS and +3 SDS at week 26 as compared to 47.4% and 35.6% using the typical approach and the “healthy” normative ranges noted above, and 27.9% and 16.3% of ThGRF-treated patients larger than +2 and +3 SDS at week 52 as compared to 33.7% and 22.6% using the typical approach and the “healthy” normative ranges noted above.

TABLE IV
Mean IGF-1 SDS Assuming Normal Distribution of IGF-1
Levels (based on Phase 3 HIV⁺ Male Patients)

LIPO-010 Main/CTR-1011

| | ThGRF | |
|---|---|---|
| | (2 mg/day) | Placebo |
| MAIN PHASE | (N = 543) | (N = 263) |
| Baseline (n, %) | | |
| SDS >2 | 14 (2.6) | 11 (4.2) |
| SDS >3 | 5 (0.9) | 6 (2.3) |
| Week 26 (n, %) | | |
| SDS >2 | 167 (41.2) | 6 (30) |
| SDS >3 | 103 (25.4) | 1 (0.5) |

| | LIPO-010 Extension/CTR-1012 | |
|---|---|---|
| | T-T | T-P |
| EXTENSION PHASE | (N = 246) | (N = 135) |
| Week 27 (n, %) | | |
| SDS >2 | 105 (44.5) | 53 (40.2) |
| SDS >3 | 69 (29.2) | 28 (21.2) |
| Week 52 (n, %) | | |
| SDS >2 | 53 (27.9) | 1 (1.1) |
| SDS >3 | 31 (16.3) | 0 (0.0) |

T-T = ThGRF (2 mg/day) for week 1 to 26 - ThGRF (2 mg/day) for week 27 to 52;
T-P = ThGRF (2 mg/day) for week 1 to 26 - Placebo for week 27 to 52.

The calculations above (based on “healthy”, typical normative ranges or using normative ranges based on IGF-1 levels in HIV subjects) were all done assuming a Normal distribution of the IGF-1 levels. However, it was noted that the distribution of the IGF-1 levels at baseline for the patients in the phase 3 clinical studies is “Lognormal” rather than “Normal”. Thus, the means and standard deviations were recalculated using the natural log(logₑ or In) of IGF-1 and then calculated IGF-1 SDS using the lognormal scale, i.e. [(natural log IGF-1) minus (mean) divided by (standard deviation)]. The results using lognormal distributions are presented in Table V for the main phase and the extension phase. Importantly, the percent of patients at baseline and those treated with placebo is now much closer to expected and in addition, the % of ThGRF-treated patients being larger than +2 and +3 SDS using the lognormal distribution and the HIV population normative ranges are substantially lower than that seen using the uncorrected SDS calculators.

TABLE V
Mean IGF-1 SDS Using Log-Normal Distribution
(Based on Phase 3 HIV⁺ Male Patients)

LIPO-010 Main/CTR-1011

| | ThGRF | |
|---|---|---|
| | (2 mg/day) | Placebo |
| MAIN PHASE | (N = 543) | (N = 263) |
| Baseline (n, %) | | |
| SDS >2 | 7 (1.3) | 8 (3.1) |
| SDS >3 | 1 (0.2) | 3 (1.1) |
| Week 26 (n, %) | | |
| SDS >2 | 122 (30.1) | 2 (1.0) |
| SDS >3 | 30 (7.4) | 1 (0.5) |
| | LIPO-010 Extension/CTR-1012 | |
| | T-T | T-P |
| EXTENSION PHASE | (N = 246) | (N = 135) |
| Week 27 (n, %) | | |
| SDS >2 | 77 (32.6) | 38 (28.8) |
| SDS >3 | 18 (7.6) | 7 (5.3) |
| Week 52 (n, %) | | |
| SDS >2 | 37 (19.5) | 0 (0.0) |
| SDS >3 | 11 (5.8) | 0 (0.0) |

T-T = ThGRF (2 mg/day) for week 1 to 26 - ThGRF (2 mg/day) for week 27 to 52;
T-P = ThGRF (2 mg/day) for week 1 to 26 - Placebo for week 27 to 52.

As such, described herein is an improved, more statistically valid, approach for determining the normative range of IGF-1 in HIV subjects, using IGF-1 values measured in HIV-subjects (rather than non-HIV subjects), with a logarithmic transformation. This novel approach represents an important new tool for clinicians caring for HIV patients, for example to assess dose adjustments of GH stimulation therapy monitored via IGF-1 levels or drug continuation/discontinuation. The new upper limit of normal (97%) that is estimated by this calculation could serve as a benchmark for possible dose titration or drug continuation/discontinuation.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, an and “the” include corresponding plural references unless the context clearly dictates otherwise.

1. (canceled)

2. A method for determining whether an HIV-infected subject has a normal insulin-like growth factor 1 (IGF-1) level, said method comprising:
(a) obtaining an IGF-1 value from a blood-derived sample from said HIV-infected subject; and
(b) determining an IGF-1 standard deviation score (SDS) based on said IGF-1 value, wherein said IGF-1 SDS is determined using the following equation:
IGF-1 SDS=(x−μ)/σ
in which
x is the log of said IGF-1 value;
μ is the mean of log of IGF-1 values obtained in blood-derived samples from a population of age- and gender-matched HIV-infected subjects; and
σ is the standard deviation of said log of IGF-1 values obtained in blood-derived samples from a population of age- and gender-matched HIV-infected subjects;
wherein an IGF-1 SDS determined by said equation that is ≧−z_{α} or ≦z_{α} is indicative that said HIV-infected subject has a normal IGF-1 level, and wherein an IGF-1 SDS determined by said equation that is <−z_{α} or >z_{α} is indicative that said HIV-infected subject has an abnormal IGF-1 level, wherein z_{α} is from 1.282 to 5.0.

3. (canceled)

4. The method of claim 2, wherein said log is logₑ.

5. The method of claim 2, wherein z_{α} is 1.645, 1.96, 2.0, 2.5, 2.576 or 3.0.

6-10. (canceled)

11. The method of claim 2, wherein said IGF-1 level is a serum IGF-1 level and said blood-derived sample is a serum sample.

12. (canceled)

13. A method for determining whether an HIV-infected subject has a normal insulin-like growth factor 1 (IGF-1) level, said method comprising:
(a) obtaining an IGF-1 value from a blood-derived sample from said HIV-infected subject;
(b) performing a log transformation of said IGF-1 value to obtain a log transformed IGF-1 value; and
(c) comparing said log transformed IGF-1 value to a log transformed normative range, wherein said log transformed normative range is determined using the following equation:
log transformed normative range=μ±(z_{α}σ)
in which
μ is the mean of log_{A} transformed IGF-1 values obtained in blood-derived samples from a population of age- and gender-matched HIV-infected subjects;
A is the base of log_{A};
z_{α} is from 1.282 to 5.0; and
σ is the standard deviation of said log_{A} of IGF-1 values obtained in blood-derived samples from a population of age- and gender-matched HIV-infected subjects;
wherein a log_{A} transformed IGF-1 value that is within said log_{A} transformed normative range is indicative that said HIV-infected subject has a normal IGF-1 level, and a log_{A} transformed IGF-1 value that is outside said log_{A} transformed normative range is indicative that said HIV-infected subject has an abnormal IGF-1 level.

14. (canceled)

15. A method for determining whether an HIV-infected subject has a normal insulin-like growth factor 1 (IGF-1) level, said method comprising:
(a) obtaining an IGF-1 value from a blood-derived sample from said HIV-infected subject; and
(b) comparing said IGF-1 value to a normative range, wherein said normative range is determined using the following equation:
normative range=A(μ±(Z_{α}·σ))
in which
μ is the mean of log_{A} transformed IGF-1 values obtained in blood-derived samples from a population of age- and gender-matched HIV-infected subjects;
A is the base of log_{A};
z_{α} is from 1.282 to 5.0; and
σ is the standard deviation of said log_{A} of IGF-1 values obtained in blood-derived samples from a population of age- and gender-matched HIV-infected subjects;
wherein an IGF-1 value that is within said normative range is indicative that said HIV-infected subject has a normal IGF-1 level, and an IGF-1 value that is outside said normative range is indicative that said HIV-infected subject has an abnormal IGF-1 level.

16. (canceled)

17. The method of claim 15, wherein A is e.

18. The method of claim 13, wherein z_{α} is 1.645, 1.96, 2.0, 2.5, 2.576 or 3.0.

19-23. (canceled)

24. The method of claim 13, wherein said IGF-1 level is serum IGF-1 level and said blood-derived sample is a serum sample.

25. The method of claim 2, wherein said HIV-infected subject suffers from lipodystrophy.

26. The method of claim 2, wherein said HIV-infected subject is undergoing a growth hormone stimulation therapy.

27. A method for determining a normative range for the monitoring of insulin-like growth factor 1 (IGF-1) levels in HIV-infected subjects, said method comprising:
(a) obtaining IGF-1 values from blood-derived samples from a population of HIV-infected subjects;
(b) calculating said normative range in accordance with the following equation:
normative range=A(μ±(Zα·σ))
in which
μ is the mean of log A transformed IGF-1 values obtained in blood-derived samples from a population of HIV-infected subjects;
A is the base of log_{A};
z_{α} is from 1.282 to 5.0; and
σ is the standard deviation of said log_{A} of IGF-1 values obtained in blood-derived samples from a population of HIV-infected subjects.

28. (canceled)

29. The method of claim 27, wherein log_{A} is logₑ.

30. The method of claim 27, wherein z_{α} is 1.645, 1.96, 2.0, 2.5, 2.576 or 3.0.

31-35. (canceled)

36. The method of claim 27, wherein said IGF-1 levels are serum IGF-1 levels and said blood-derived samples are serum samples.

37. The method of claim 27, wherein said HIV-infected subject suffers from lipodystrophy.

38. (canceled)

39. A method of monitoring GH stimulation therapy in an HIV-infected subject, comprising
(i) determining whether the subject exhibits a normal IGF-1 level using the method of claim 2, wherein:
(a) a higher than normal IGF-1 level is indicative that the GH stimulation therapy is resulting in a GH level that is higher than a normal GH level;
(b) a lower than normal IGF-1 level is indicative that the GH stimulation therapy is resulting in a GH level that is lower than a normal GH level; and
(c) an IGF-1 level falling within a normative range is indicative that the GH stimulation therapy is resulting in a normal GH level; and
(ii) adjusting or modifying the GH stimulation therapy in the subject undergoing GH stimulation therapy in accordance with the determination of whether the subject exhibits a normal IGF-1 level.

40. A method of determining whether GH stimulation therapy of an HIV subject should be adjusted or modified, comprising
(i) determining whether the subject exhibits a normal IGF-1 level using the method of claim 2, wherein:
(a) a higher than normal IGF-1 level is indicative that the GH stimulation therapy should be reduced, interrupted or stopped;
(b) a lower than normal IGF-1 level is indicative that the GH stimulation therapy should be increased; and
(c) an IGF-1 level falling within a normative range is indicative that the GH stimulation therapy may be maintained without any significant adjustment;
(ii) adjusting or modifying the GH stimulation therapy in the subject undergoing GH stimulation therapy in accordance with the determination of whether the subject exhibits a normal IGF-1 level.

41. (canceled)

42. A program storage device readable by an electronic medium and tangibly storing instructions executable by the electronic medium to perform step (b) of the method of claim 2 and provide an indication as to whether the subject has a normal IGF-1 level.

43. A computer program product comprising a computer useable medium that tangibly stores as computer readable code instructions to perform step (b) of the method of claim 2 and provide an indication as to whether the subject has a normal IGF-1 level.

44-47. (canceled)
